(19) United States
(12) Patent Application Publication
(10) Pub. No.: US 2022/0340985 A1
(43) Pub. Date: Oct. 27, 2022

(54) MATERIALS AND METHODS FOR DETECTING HUMAN PAPILLOMA VIRUS

(71) Applicant: UNIVERSITY OF MIAMI, Miami, FL (US)

(72) Inventors: Jean-Marc Zingg, Miami, FL (US); Pratibha Mahesh Joshi, Miami, FL (US); Eric Kobetz, Miami, FL (US); Sapna Deo, Miami, FL (US); Sylvia Daunert, Miami, FL (US)

(21) Appl. No.: 17/761,064

(22) PCT Filed: Sep. 15, 2020
(86) PCT No.: PCT/US20/50926

§ 371 Date: Mar. 16, 2022

Related U.S. Application Data
(60) Provisional application No. 62/947,127, filed on Dec. 12, 2019.
(60) Provisional application No. 62/901,662, filed on Sep. 17, 2019.

(51) Int. Cl. C12Q 1/70; C12Q 1/6848; B01L 3/00

(52) U.S. Cl. CPC C12Q 1/708; C12Q 1/6848; B01L 3/5023; B01L 2300/0825

(57) ABSTRACT

The disclosure relates to test kits and methods for detecting the presence of multiple human papilloma vims polynucleotides in a biological sample.

### CROSS-REFERENCE TO RELATED APPLICATION

The benefit under 35 U.S.C. §0 119(e) of both U.S. Provisional Patent Application No. 62/901,662, filed Sep. 17, 2019, and U.S. Provisional Patent Application No. 62/947,127 filed Dec. 12, 2019, are hereby claimed, the disclosures of which are incorporated herein by reference in their entireties.

### FIELD OF THE INVENTION

The disclosure relates to test kits and methods for detecting the presence of multiple human papilloma virus polynucleotides in a biological sample.

### INCORPORATION BY REFERENCE

This application contains, as a separate part of the disclosure, a sequence listing in computer-readable form (filename: 54789_SeqListing.txt, 4979 bytes, created Sep. 15, 2020), which is incorporated by reference in its entirety.

### BACKGROUND

With the original recognition of human papilloma virus (HPV) as an important oncovirus for several types of cancer, the early and reliable detection of HPV has become key for successful therapy (1, 2). For cervical cancer, which is by more than 95% due to high-risk HPV (hrHPV) types infection (3-6), the current prophylactic vaccination against 4-9 hrHPV types may lower the disease incidence; however, this may not occur in populations with insufficient resources and compliance (7). The detection of hrHPV is complicated by the fact that over 240 HPV variants have been found so far, with only a subset of 14 hrHPV types leading to cervical cancer (8). To date, whereas several methods of hrHPV detection have been developed for laboratory use, only seven are currently FDA approved and used for diagnosis of hrHPV in the clinic (9). However, all approved methods require well-equipped laboratories with electricity, expensive equipment and trained personal, and therefore are not useful for primary screening in a low-cost point of care setting and/or for self-diagnosis (10).

As for point of care detection of other pathogens, a myriad of methods able to detect hrHPV with various efficiency, sensitivity, accuracy, time and need for equipment have been and are currently developed (11, 12). Amongst the isothermal methods (13-19), the recombinase polymerase amplification technique (RPA) offers rapid amplification and high specificity and sensitivity similar to the polymerase chain reaction (PCR) (20). Since its first demonstrations in 2006, the RPA method has been further developed and used in many applications to rapidly (10-20 min) amplify DNA and RNA for subsequent detection and diagnosis of pathogens (e.g., bacteria, virus, parasites, and cancer cells) (20). Several strategies have been developed for detecting the amplified products on agarose gels, paper-based lateral flow assay and even in real-time (analogous to real-time PCR) (20). Detection limits as low as 1-100 copies were reported for RPA and the method is relatively tolerant to contaminants often present in samples (blood, serum, mucosa, etc.) (20). The RPA components can be assembled in a few steps, and they can be purchased from the manufacturer in lyophilized form making it suitable for use in remote locations.

### SUMMARY

In one aspect, described herein is a kit for the detection of high risk human papilloma virus (hrHPV) polynucleotides in a biological sample comprising (a) a primer pair; and (b) a detection probe; wherein (a) and (b) are capable of detecting the presence of each of the hrHPV polynucleotides, if present, in the sample by recombinase polymerase amplification (RPA). Exemplary hrHPV include, but are not limited to, hrHPV is HPV16, HPV18, HPV35, HPV31, HPV33, HPV39, HPV45, HPV51, HPV52, HPV56, HPV58, HPV59, HPV66 and HPV68. In some embodiments, the primer pair is capable of amplifying each of HPV16, HPV18, HPV35, HPV31, HPV33, HPV39, HPV45, HPV51, HPV52, HPV56, HPV58, HPV59, HPV66 and HPV68 in a biological sample.

In some embodiments, the primer pair comprises the nucleotide sequence set forth in SEQ ID NOs: 9 and 11. In some embodiments, the primer pair comprises the nucleotide sequence set forth in SEQ ID NOs: 10 and 11. In some embodiments, the primer pair comprises the nucleotide sequence set forth in SEQ ID NOs: 12 and 13. In some embodiments, the primer pair comprises a first primer that is phosphorylated at the 5′-end and a second primer that is labeled with 6-carboxyfluorescein (FAM). In some embodiments, the nucleotide sequence set forth in SEQ ID NO: 9 is phosphorylated and the nucleotide sequence set forth in SEQ ID NO: 11 is labeled with 6-carboxyfluorescein (FAM). In some embodiments, the detection probe comprises a nucleotide sequence set forth in SEQ ID NO: 14 (HPVDET1) or SEQ ID NO: 23 (HPVDET11). In some embodiments, the detection probe is biotinylated.

In some embodiments, the kit further comprises a running buffer and optionally a test strip (e.g., filter paper (e.g., with capture line with streptavidin) or chitosan). In some embodiments, the running buffer comprises magnesium chloride and sodium chloride. In some embodiments, running buffer comprises about 420 mM sodium chloride, and about 83 mM magnesium chloride, pH 6.5-8.5.

Also described herein is a method for detecting high risk human papilloma virus (hrHPV) polynucleotides in a biological sample comprising: (a) an amplifying step comprising adding the biological sample to a vessel containing a primer pair that is capable of each of the hrHPV polynucleotides, if present, in the biological sample, (b) digesting amplified hrHPV polynucleotides in the vessel into a single-stranded amplified product; (c) combining the single-stranded amplified product with a running buffer comprising a detection probe that is capable of detecting the single-stranded amplified product to form a mixture, and incubating the mixture for a period of time in the vessel; and, (d) a detecting step comprising wicking the mixture into a test strip and visually detecting the detection probe on the test strip. In some embodiments, the digesting step comprises adding an exonuclease to the vessel before the detecting step. In some embodiments, the exonuclease is lambda exonuclease.

In some embodiments, the amplifying step does not comprise incubating the mixture at a temperature greater than about 37° C. In some embodiments, the amplifying step does not comprise incubating the mixture at a temperature greater than about 42° C. In some embodiments, the amplifying step comprises incubating the mixture for about 10 minutes to about 2 hours (e.g., 10 minutes, 20 minutes, 30 minutes, 40 minutes, 50 minutes, 60 minutes, 70 minutes, 80 minutes, 90 minutes, 100 minutes, 110 minutes or 120 minutes).

The foregoing summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE FIGURES

FIG. 1 provides an image of paper test strips and table showing the limit of detection with specific recombinase polymerase amplification (RPA) primers (Biotin-F12 and FAM-R7).

FIG. 2 provides a gel and an image of paper test strips showing that recombinase polymerase amplification (RPA) with labeled primers gives background on a lateral flow assay.

FIG. 3 depicts the generation of FAM-labelled single stranded recombinase polymerase amplification (RPA) products and detection after digestion by lambda exonuclease by biotinylated detection probes (e.g., HPVDET, SEQ ID NO: 14 or SEQ ID NOs: 14-23).

FIG. 4 provides a gel and an image of paper test strips showing the optimization of primers for recombinase polymerase amplification (RPA) and subsequent detection of hrHPV16 by lateral flow assay.

FIG. 5 provides an image of paper test strips showing results of the isothermal rapid hrHPV method. Plasmid samples (100 ng) of all 14 hrHPV types and one clinical sample (HPV35) were amplified with recombinase polymerase amplification (RPA) and consensus primer pair p-IPf/FAM-IPr (SEQ ID NO: 9 and SEQ ID NO: 11 (5′-phosphorylated or labeled at the 5′ end with 6-carboxyfluorescein (FAM), respectively, and detected with lateral flow assay using a biotinylated universal detection probe (HPVDET1, SEQ ID NO: 14) (HPV66 is not shown).

FIG. 6 provides an image of paper test strips and table showing the limit of detection with consensus RPA primers and universal detection probe. Serial dilution of a plasmid containing HPV35 (pHPV35), amplification by RPA and primers p-IPf and FAM-IPr SEQ ID NO: 9 and SEQ ID NO: 11 (5′-phosphorylated or labeled at the 5′ end with 6-carboxyfluorescein (FAM), respectively, digestions with lambda exonuclease and detection with lateral flow assay with biotinylated HPVDET1 (SEQ ID NO: 14).

FIG. 7 provides an image of paper test strips and gel showing the detection of hrHPV with alternative consensus RPA primers and universal detection probe HPVDET1 (SEQ ID NO: 14). Amplification of plasmids containing HPV16 and HPV35 or clinical sample (HPV35) by RPA and primers p-IP2f and FAM-IPr (SEQ ID NO: 9 and SEQ ID NO: 11 (5′-phosphorylated or labeled at the 5′ end with 6-carboxyfluorescein (FAM), respectively), separation by 2% agarose gel, or digestion with lambda exonuclease and detection with lateral flow assay with biotinylated HPVDET1 (SEQ ID NO: 14).

FIG. 8 provides a ClustalW sequence alignment of the 14 high-risk and 5 low-risk HPV types.

### DETAILED DESCRIPTION

The present disclosure provides kits and methods for detecting human papilloma virus (HPV) in biological samples that eliminate the need for laboratory equipment. The kits and methods provide a solution to diagnosing infection and managing public health in medically underserved populations worldwide, while specifically addressing health challenges faced by developing communities.

Current commercial methods of detection of high-risk human papilloma virus (hrHPV) rely mostly on the polymerase chain reaction (PCR) that use either 14 specific primer pairs or a single consensus primer pair (e.g. GPS+/GP6+). However, PCR requires the use of a thermal cycler (PCR machine) and electricity and therefore is not suitable for a low-cost Point of Care Test (PoCT). Moreover, these PCR primers are not suitable for isothermal amplification since at lower temperature they may anneal non-specifically leading to background amplification.

To date, whereas several methods of hrHPV detection have been developed for laboratory use, only seven are currently FDA approved and used for diagnosis of hrHPV in the clinic (9).

Described herein is an isothermal point of care method for the rapid detection of hrHPV that works at a constant temperature (<42° C.) using recombinase polymerase amplification (RPA). RPA operates isothermally at temperatures between 25° C. and 42° C. and to prevent primer dimer formation and non-specific background amplification requires careful selection of primers and conditions for optimal amplification (20, 21). Since RPA is based on homologous recombination, the primers may need to anneal without mismatches to their targets for optimal performance, suggesting that for 14 hrHPV types 28 primers will be required (e.g. as reported for a two-stage multiplexed detection system) (22-24). However, a limited number of mismatches is tolerated for RPA primers but it depends on their position within the primer and it can reduce the amplification efficiency (25). The polynucleotide amplification reactions can be run at constant temperatures (e.g., staying within five degrees from the starting temperature) that are near ambient or room temperature, for example, about 20° C. to about 37° C.

Also described herein are universal primer pairs that are capable of amplifying all 14 hrHPV types (e.g., HPV16, HPV18, HPV35, HPV31, HPV33, HPV39, HPV45, HPV51, HPV52, HPV56, HPV58, HPV59, HPV66, HPV68). To enable the detection of the amplified double-stranded RPA products, one primer was labelled with a 5′-phosphate, and the other primer was labelled at the 5′-end with 6-carboxyfluorescein (FAM). Then, the double-stranded labelled RPA product was digested with lambda exonuclease, which preferentially digests 5′-phosphorylated DNA ends whereas the 5′-FAM ends are protected, generating single-stranded 5′-FAM-labelled RPA products. These products can be detected by paper-based lateral flow assays (LFA) using biotinylated detection probes that are either specific to each hrHPV type or detect all 14 hrHPV types at the same time (universal consensus detection probe). Lateral flow assay conditions were developed that allow these probes to detect all 14 hrHPV types even at room temperature. Using this isothermal rapid hrHPV detection method, amplification of all 14 hrHPV types at constant temperature (e.g., at about 37° C.) within short time (e.g., less than 1 hour) was achieved.

The present disclosure identifies the recombinase polymerase amplification (RPA) method as being suitable for the isothermal detection of the 14 hrHPV types in a low-cost point of care setting. As described in the Examples, using plasmid templates, genomic DNA from hrHPV infected cells and clinical samples, several specific primer pairs were generated that were able to amplify specific hrHPV types by RPA. Unexpectedly, consensus primer pairs could also be designed able to amplify all 14 hrHPV types. The method and assay conditions were adapted for subsequent detection of the RPA products by lateral flow assay on paper dipsticks using universal consensus detection probes. The isothermal rapid hrHPV detection method does not need costly instrumentation, trained personal and cumbersome multi-step protocols and facilitates self-sampling and “on-site” detection of hrHPV.

After sample collection, a simple extraction, purification, enrichment and concentration step can be used to increase the sensitivity of the assay.

The amplification reactions and digestion by lambda exonuclease can be run in a single reaction vessel. The test strip serves as a separation device that detects labelled detection probes hybridized to amplified hrHPV nucleic acids based on capture probes embedded in the test strip. Because the amplification can be completed in as little as 10-30 minutes, virtually immediate results can be provided on-site, rather than requiring days or weeks for results to be returned from a clinician or laboratory.

Detection Methods

In one aspect, the disclosure provides a method for detecting a high risk human papilloma virus (hrHPV) polynucleotide in a biological sample comprising an amplifying step comprising adding the biological sample to a vessel containing a primer pair that is capable of amplifying each of the hrHPV polynucleotides, if present, in the biological sample, digesting amplified hrHPV into a single stranded amplified product; combining the amplified product with a running buffer comprising a detection probe that is capable of detecting the single-stranded amplified product, and incubating the mixture for a period of time in the vessel; and a detecting step comprising wicking the mixture onto a test strip and visually detecting the detection probe on the test strip.

In some embodiments, the digesting step further comprises adding an exonuclease to the vessel before the detecting step to generate a single stranded amplified product. In some embodiments, wherein the exonuclease is lambda exonuclease.

In some embodiments, the digesting step does not comprise adding a exonuclease to the vessel before the detection step. In such embodiments, single-stranded amplified products can also be generated using tailed primers with an internal C3 spacer (3 hydrocarbons) (Jauset-Rubio et al., Scientific Reports 6:37732, 2016, the disclosure of which is incorporated by reference in its entirety). Such primers contain an additional sequence at the 5′-end separated by a C3 spacer from the sequence required for amplification. This additional sequence will not be copied during RPA since the C3 spacer blocks the polymerase. Hence, the single stranded amplified product can be detected by hybridizing a detection probe and subsequent lateral flow assay.

In some embodiments, the primer pair comprises a nucleotide sequence that is at least 90% identical (e.g., at least 90%, 91%, 92%, 93%, 94%, 95%, 96%, 97%, 98%, 99% or more identical) to a nucleotide sequence set forth in SEQ ID NOs: 7-13. In some embodiments, the primer comprises additional nucleotides at either the N-terminus or C-terminus of the nucleotide sequence set forth in SEQ ID NO: 7-13. For example, in some embodiments, the primer comprises 1, 2, 3, 4, 5, 6, 7, 8, 9 or 10 additional nucleotides at the N-terminus of SEQ ID NOs: 7-13. In some embodiments, the primer comprises 1, 2, 3, 4, 5, 6, 7, 8, 9 or 10 additional nucleotides at the C-terminus of SEQ ID NOs: 7-13. In some embodiments, the primer pair comprises a nucleotide sequence set forth in SEQ ID NOs: 7-13. In some embodiments, the primer comprises a nucleotide sequence that is 20, 21, 22, 23, 24, 25, 26, 27, 28, 29, 30, 31, 32, 33, 34 or 35 nucleotides in length. In some embodiments, the primer pair comprises any combination of two primers comprises a nucleotide sequence set forth in SEQ ID NOs: 7-13. In some embodiments, the primer pair comprises the nucleotide sequences set forth in SEQ ID NOs: 5 and 6, or SEQ ID NOs: 7 and 8, or SEQ ID NOs: 9 and 11, or SEQ ID NOs: 10 and 11, or SEQ ID NOs: 12 and 13. In some embodiments, the primer pair comprises the nucleotide sequence set forth in SEQ ID NOs: 9 and 11. In some embodiments, the nucleotide sequence set forth in SEQ ID NO: 9 is phosphorylated and the nucleotide sequence set forth in SEQ ID NO: 11 is labeled (e.g., with 6-carboxyfluorescein (FAM)). In some embodiments, the nucleotide sequence set forth in SEQ ID NO: 9 is phosphorylated and the nucleotide sequence set forth in SEQ ID NO: 10 is labeled (e.g., with 6-carboxyfluorescein (FAM)). In some embodiments, the nucleotide sequence set forth in SEQ ID NO: 9 is phosphorylated and the nucleotide sequence set forth in SEQ ID NO: 12 is labeled (e.g., with 6-carboxyfluorescein (FAM)).

In some embodiments, the detection probe comprises nucleotide sequence that is at least 90% identical (e.g., at least 90%, 91%, 92%, 93%, 94%, 95%, 96%, 97%, 98%, 99% or more identical) to a nucleotide sequence set forth in any one of SEQ ID NOs: 14, 19, and 21-23. In some embodiments, the detection probe comprises a nucleotide sequence set forth in any one of SEQ ID NOs: 14, 19 and 21-23. In some embodiments, the detection probe comprises additional nucleotides at either the N-terminus or C-terminus of the nucleotide sequence set forth in SEQ ID NO: 14, 19 and 21-23. For example, in some embodiments, the detection probe comprises 1, 2, 3, 4, 5, 6, 7, 8, 9 or 10 additional nucleotides at the N-terminus of SEQ ID NOs: 14, 19 and 21-23. In some embodiments, the detection probe comprises 1, 2, 3, 4, 5, 6, 7, 8, 9 or 10 additional nucleotides at the C-terminus of SEQ ID NOs: 14, 19 and 21-23. In some embodiments, the detection probe comprises a nucleotide sequence set forth in SEQ ID NO: 14 or 23. In some embodiments, the detection probe comprises the nucleotide sequence set forth SEQ ID NO: 14. In some embodiments, the detection probe is biotinylated. In some embodiments, the detection probe is biotinylated and comprises the nucleotide sequence set forth in SEQ ID NO: 14.

In some embodiments, the primer pair comprises SEQ ID NOs: 9 and 11 and the detection probe comprises SEQ ID NO: 14.

The biological sample is, in various embodiments, obtained from a human or other mammalian subject, for example, by collecting a bodily fluid sample or swabbing a body orifice. The sample may be collected by, e.g., a health care worker or self-sampling. Alternatively, the biological sample is obtained from an environmental source, such as a water or soil or a contaminated surface, device or laboratory equipment. The biological sample may also be a food sample (e.g., a fluid or swab taken from food in order to, for example, detect contamination).

When the mixture of the biological sample and reagents is formed, if an hrHPV polynucleotide is present in the biological sample, RPA amplification occurs and the detection probe hybridizes to the single-stranded copies of the amplified product to form a reporter complex.

Optionally, the detection probe is conjugated to a microparticle. The term “microparticle” refers to a particle comprising a diameter less than 100 micrometers and includes particles having a diameter less than one micrometer. Microparticles may be spherical (e.g., microbeads) or have an irregular shape, and may be composed of any of a number of substances, including gold and/or other metals, nylon and/or other polymers, magnetic compounds, and combinations thereof. In one aspect, the microparticle has a diameter less than about one micrometer. In various aspects, the microparticle is selected from a nylon microparticle, gold microparticle, or magnetic (e.g., paramagnetic) microparticle. Combinations of microparticles may also be used. Conjugation of the detection probe and microparticle can be achieved using any suitable method, such as covalent linkage. The labelled detection probe can be detected using lateral flow assay or by any other method suitable to detect it.

The running buffer for the detection by lateral flow assay, in one aspect, comprises magnesium chloride and sodium chloride, optionally about 1 mM to about 100 mM magnesium chloride and about 1 mM to about 500 mM sodium chloride. In some embodiments, the running buffer comprises magnesium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM or about 100 mM. In some embodiments, the running buffer comprises sodium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 100 mM, about 200 mM, about 300 mM, about 400 mM, about 410 mM, about 415 mM, about 420 mM, about 425 mM, about 430 mM, about 435 mM, about 440 mM, about 445 mM, about 450 mM, about 455 mM, about 460 mM, about 465 mM, about 470 mM, about 475 mM, about 480 mM, about 485 mM, about 490 mM, about 495 mM, or about 500 mM. For example, in some embodiments, a 1× running buffer comprises about 83 mM magnesium chloride and about 420 mM sodium chloride, between pH 6.5 and 8.5. In one aspect, the amplification step is performed at a temperature of less than about 42° C. (e.g., between about 25° C. and 42° C.), unlike traditional PCR reactions, which require laboratory equipment for temperature cycling to achieve temperatures greater than 90° C. Therefore, in one aspect, the amplification step does not comprise incubating the mixture at a temperature greater than about 42° C. In one aspect, the amplification step comprises incubating the mixture at a temperature between about 20° C. and about 42° C., for example, between about 22° C. and about 35° C., between about 23° C. and about 32° C., or between about 25° C. and about 30° C. In another aspect, the amplification step does not comprise incubating the mixture at a temperature greater than about 30° C. Optionally, the amplification step comprises incubating the mixture at a constant temperature of about 37° C. The term “constant temperature” refers to temperatures that are within ±5° C. of a reference temperature. In some embodiments, the amplification step comprises incubating the mixture at a temperature of about 37° C. or less for a time of about 10 minutes to about 2 hours, for example, about 10 minutes, about 20 minutes, about 30 minutes, about 40 minutes, about 50 minutes, about 60 minutes, about 90 minutes or about 2 hours. In some embodiments, the amplification step comprises incubating the mixture at a temperature of about 37° C. or less for a time of about 10 minutes to about 20 minutes.

In one aspect, a method of the disclosure further comprises a detection step comprising wicking the mixture, e.g., via capillary action, into a test strip and visually detecting the detection probe. In some embodiments, the test strip is a paper strip, optionally comprising filter paper, such as Whatman #1 filter paper. The test strip optionally comprises pores having a diameter of about 5 micrometers to about 20 micrometers, for example, about 5 micrometers, about 10 micrometers, about 11 micrometers, about 12 micrometers, about 13 micrometers, about 14 micrometers, about 15 micrometers, or about 20 micrometers. Optionally, the test strip comprises a region comprising a capture line of streptavidin, or chitosan, which non-specifically binds polynucleotides and provides a control region or indicator of test completion. The test strip separates the components in the mixture based on size exclusion so that reporter probes hybridized to amplified polynucleotides, i.e., the reporter complexes, travel less along the length of the test strip than smaller, uncomplexed reporter probes. Thus, when a hrHPV polynucleotide is present in the biological sample, a distinct band is visible near the bottom of the test strip, e.g., below an indicator of test completion, indicating that a hrHPV polynucleotide is present in the biological sample. In contrast, a test strip dipped into a mixture containing only uncomplexed reporter probes exhibits a band farther up the test strip, e.g., at the mid-point of the strip or at an indicator of test completion, indicating that a hyHPV polynucleotide is not present in the biological sample.

Kits

The disclosure also provides a kit for the detection of one or more high risk human papilloma virus (hrHPV) polynucleotides in a biological sample, the kit comprising a primer pair and a biotinylated detection probe, wherein the primer pair and the detection probe are capable of detecting each of the hrHPV polynucleotides, if present, in the sample. In some embodiments, the hrHPV polynucleotide is a polynucleotide from HPV16, HPV18, HPV35, HPV31, HPV33, HPV39, HPV45, HPV51, HPV52, HPV56, HPV58, HPV59, HPV66 or HPV68. In some embodiments, the primer pair and the detection probe are capable of identifying two, three, four, five, six, seven, eight, nine, ten, eleven, twelve, thirteen or all fourteen hrHPV genotypes.

In some embodiments, the primer pair comprises a nucleotide sequence that is at least 90% identical (e.g., at least 90%, 91%, 92%, 93%, 94%, 95%, 96%, 97%, 98%, 99% or more identical) to a nucleotide sequence set forth in SEQ ID NOs: 7-13. In some embodiments, the primer comprises additional nucleotides at either the N-terminus or C-terminus of the nucleotide sequence set forth in SEQ ID NO: 7-13. For example, in some embodiments, the primer comprises 1, 2, 3, 4, 5, 6, 7, 8, 9 or 10 additional nucleotides at the N-terminus of SEQ ID NOs: 7-13. In some embodiments, the primer comprises 1, 2, 3, 4, 5, 6, 7, 8, 9 or 10 additional nucleotides at the C-terminus of SEQ ID NOs: 7-13. In some embodiments, the primer comprises a nucleotide sequence that is 20, 21, 22, 23, 24, 25, 26, 27, 28, 29, or 30 nucleotides in length. In some embodiments, the primer pair comprises a nucleotide sequence set forth in SEQ ID NOs: 7-13. In some embodiments, the primer pair comprises any combination of two primers comprises a nucleotide sequence set forth in SEQ ID NOs: 7-13. In some embodiments, the primer pair comprises the nucleotide sequences set forth in SEQ ID NOs: 5 and 6, or SEQ ID NOs: 7 and 8, or SEQ ID NOs: 9 and 11, or SEQ ID NOs: 10 and 11, or SEQ ID NOs: 12 and 13. In some embodiments, the primer pair comprises the nucleotide sequence set forth in SEQ ID NOs: 9 and 11. In some embodiments, the nucleotide sequence set forth in SEQ ID NO: 9 is phosphorylated and the nucleotide sequence set forth in SEQ ID NO: 11 is labeled (e.g., with 6-carboxyfluorescein (FAM)). In some embodiments, the nucleotide sequence set forth in SEQ ID NO: 9 is phosphorylated and the nucleotide sequence set forth in SEQ ID NO: 10 is labeled (e.g., with 6-carboxyfluorescein (FAM)). In some embodiments, the nucleotide sequence set forth in SEQ ID NO: 9 is phosphorylated and the nucleotide sequence set forth in SEQ ID NO: 12 is labeled (e.g., with 6-carboxyfluorescein (FAM)).

In some embodiments, the detection probe comprises nucleotide sequence that is at least 90% identical (e.g., at least 90%, 91%, 92%, 93%, 94%, 95%, 96%, 97%, 98%, 99% or more identical) to a nucleotide sequence set forth in any one of SEQ ID NOs: 14, 19, and 21-23. In some embodiments, the detection probe comprises a nucleotide sequence set forth in any one of SEQ ID NOs: 14, 19 and 21-23. In some embodiments, the detection probe comprises additional nucleotides at either the N-terminus or C-terminus of the nucleotide sequence set forth in SEQ ID NO: 14, 19 and 21-23. For example, in some embodiments, the detection probe comprises 1, 2, 3, 4, 5, 6, 7, 8, 9 or 10 additional nucleotides at the N-terminus of SEQ ID NOs: 14, 19 and 21-23. In some embodiments, the detection probe comprises 1, 2, 3, 4, 5, 6, 7, 8, 9 or 10 additional nucleotides at the C-terminus of SEQ ID NOs: 14, 19 and 21-23. In some embodiments, the detection probe comprises a nucleotide sequence set forth in SEQ ID NO: 14 or 23. In some embodiments, the detection probe comprises the nucleotide sequence set forth SEQ ID NO: 14. In some embodiments, the detection probe is biotinylated. In some embodiments, the detection probe is biotinylated and comprises the nucleotide sequence set forth in SEQ ID NO: 14.

In some embodiments, the primer pair comprises SEQ ID NOs: 9 and 11 and the detection probe comprises SEQ ID NO: 14.

In some embodiments, the kit further comprises a test strip. In some embodiments, the test strip is a paper strip, optionally comprising filter paper, such as Whatman #1 filter paper. The test strip optionally comprises pores having a diameter of about 5 micrometers to about 20 micrometers, for example, about 5 micrometers, about 10 micrometers, about 11 micrometers, about 12 micrometers, about 13 micrometers, about 14 micrometers, about 15 micrometers, or about 20 micrometers. Optionally, the test strip comprises a region comprising a capture line such as streptavidin, or chitosan, which non-specifically binds polynucleotides and provides a control region or indicator of test completion.

In some embodiments, the kit further comprises a running buffer for detection by lateral flow assay. In some embodiments, the running buffer comprises magnesium chloride and sodium chloride, optionally about 1 mM to about 100 mM magnesium chloride and about 100 mM to about 500 mM sodium chloride. In some embodiments, the running buffer comprises magnesium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM or about 100 mM. In some embodiments, the running buffer comprises sodium chloride at a concentration of about 1 mM, 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 81 mM, about 82 mM, about 83 mM, about 84 mM, about 85 mM, about 86 mM, about 87 mM, about 88 mM, about 89 mM, about 90 mM, about 100 mM, about 200 mM, about 300 mM, about 400 mM, about 410 mM, about 415 mM, about 420 mM, about 425 mM, about 430 mM, about 435 mM, about 440 mM, about 445 mM, about 450 mM, about 455 mM, about 460 mM, about 465 mM, about 470 mM, about 475 mM, about 480 mM, about 485 mM, about 490 mM, about 495 mM, or about 500 mM. For example, in some embodiments, a 1× running buffer comprises about 83 mM magnesium chloride and about 420 mM sodium chloride, between pH 6.5 and 8.5.

All of the U.S. patents, U.S. patent application publications, U.S. patent applications, foreign patents, foreign patent applications and non-patent publications referred to in this specification, are incorporated herein by reference, in their entireties.

From the foregoing it will be appreciated that, although specific embodiments of the invention have been described herein for purposes of illustration, various modifications may be made without deviating from the spirit and scope of the invention.

The following Examples are provided to further illustrate aspects of the disclosure, and are not meant to constrain the disclosure to any particular application or theory of operation.

### EXAMPLES

Material and Methods

Plasmid preparation: All plasmids containing genomic DNA of hrHPV (16, 18, 31, 33, 35, 39, 45, 51, 52, 56, 58, 59, 66, 68) and low-risk HPV (6, 11, 44, 53, 61) were kindly provided from the International HPV Reference Center, Karolinska Institute, Stockholm. The plasmids were transformed into E. coli NEB5α (New England Biolabs) and plasmids prepared according to a protocol provided by the manufacturer (Qiagen). The plasmid concentration was determined with a NanoVue plus Spectrophotometer (GE Healthcare), and the HPV subtype confirmed by sequencing (Genewiz).

Sample collection and preparation and PCR analysis: Patient samples were collected with a careBrush and then stored in PreservCyt solution at −80° C. (Qiagen). Written informed consent was obtained from all participants. These samples were tested using an in-house PCR method with GP5+ and GP6+ primers that detect an HPV L1 capsid gene fragment essentially as previously described (Table 1) (26).

TABLE 1
RPA and PCR primers used in this
study for HPV amplification

| Primer pairs | SEQ | |
|---|---|---|
| (F: forward: | ID | |
| R: reverse) | NO: | Description |
| F: GP5+: 5′-TTT | 1 | General PCR |
| GTTACTGTGGTAGAT | | primers |
| ACTAC-3′ | | |
| R: GP6+: 5′-GAA | 2 | |
| AAATAAACTGTAAAT | | |
| CATATTC-3′ | | |
| F: F3: 5′-FAM-A | 3 | Specific RPA |
| AACAACTATACATGA | | primers for |
| TATAATATTAGAAT- | | HPV16 |
| 3′ | | |
| R: R2: | 4 | |
| 5′-Biotin- | | |
| AACAAGACATACA | | |
| TCGACCGGTCCACCG | | |
| AC-3′ | | |
| F: F12: 5′-FAM- | 5 | Specific RPA |
| AAAAGCAAAGACATT | | primers for |
| TAGAAGAAAAAAAAC | | HPV35 |
| -3′ | | |
| R: R7: | 6 | |
| 5′-Biotin- | | |
| GCTGGACCGTCAA | | |
| TGGTATCTTCCTCCT | | |
| CC-3′ | | |
| F: exGP5+: 5′-C | 7 | Extended |
| AGTTATTTGTTACTG | | General RPA |
| TGGTAGATACTAC-3′ | | primers |
| R: exGP6+: 5′-C | 8 | |
| ACAATTGAAAAATAA | | |
| ACTGTAAATCATATT | | |
| C-3′ | | |
| F: IPf: 5′-TTAT | 9 | Intermediate |
| TTGTTACTGTGGTAG | | consensus RPA |
| ATACTAC-3′ | | primers |
| F: IP2f: 5′- GC | 10 | |
| ACAAGGTCATAATAA | | |
| TGGTATTTG-3′ | | |
| R: IPr: 5′-AATT | 11 | |
| GAAAAATAAACTGTA | | |
| AATCATATTC-3′ | | |
| F: IPfino: 5′-T | 12 | Intermediate |
| TATTTGTTACTGTGG | | consensus RPA |
| TAGATACIAC-3′ | | primers with |
| | | inosine (I) |
| R: IPrino: 5′-A | 13 | |
| ATTGAAAAATAAACT | | |
| GTAAATCAIATTC-3′ | | |

PCR amplification was performed with a Mastercycler pro Gradient PCR machine (Eppendorf) using 2 μL of patient sample, 1 μL GP5+ (50 μM), 1 μL GP6+ (50 μM), 6 μL water (RT-PCR grade water (Invitrogen)), and 10 μL KOD polymerase (KOD Hotstart Mastermix 2×, EMD Millipore). Pre-cycling was 2 min denaturation at 95° C., cycling was 10 s denaturation at 95° C., 10 s annealing at 40° C., 10 s extension at 72° C. for 40 cycles, and final 4 min elongation at 4° C. The PCR products were separated by a 2% agarose gel, the band with the correct size (approximately 150 bp) extracted with a gel extraction kit (Qiagen) and the HPV type confirmed by sequencing (Genewiz).

Isothermal recombinase polymerase amplification: Isothermal RPA amplification of HPV DNA was essentially done according to the TwistAmp Basic RPA kit (TwistDX). Briefly, 2.4 μL forward primer (10 μM) and 2.4 μL reverse primer (10 μM) (Table 1), 29.5 rehydration buffer, 5 μL of 20 ng/μL of HPV plasmids or1-5 μL of patient samples, and water (RT-PCR grade water (Invitrogen)) to a total volume of 47.5 μL were added to the freeze-dried reaction mixture and mixed by pipetting and centrifuged. The reaction was then started with 2.5 μL 280 mM Mg-Acetate and incubated for 20 min at 37° C. The RPA products were separated by a 2% agarose gel, the band with the correct size (approximately 150 bp) extracted with a gel extraction kit (Qiagen) and the HPV type confirmed by sequencing (Genewiz). Images of agarose gels containing Gel Red were acquired by an Azure Biosystems C200 Imaging system. For dilution experiments, plasmids or genomic DNA were serially diluted 1/10 with water (RT-PCR grade water (Invitrogen)) in DNA LoBind tubes (Eppendorf).

Lateral flow assay on Milenia Hybridetect Dipstick: The single-stranded FAM-labeled hrHPV RPA reaction product is spotted on the Hybridetect Dipstick, processed as described in the manufacturers' protocol (Milenia) but using the modified running buffer described in [0038] and [0057] containing the biotinylated detection probe described in Table 2, and photographed.

Detection by Lateral flow assay: To 8 microL of the RPA product, 1 μL of Lambda exonuclease (NEB) and 1 μL 10× buffer was added and incubated for 20 min at 37° C. Then, 2 to 4 μL were spotted on the Hybridetect Dipstick, processed as described in the manufacturers' protocol (Milenia Biotec), and photographed. For detection of hrHPV by lateral flow assay, the protocol was modified by adding to the running buffer (100 μL) 1 μL of either the specific or the universal consensus detection probes (5′-biotinylated, 50 μM) (Table 2), 10 μL of 5 M NaCl (final 420 mM) and 10 μL 1M MgCl₂ (83 mM), and mixed by vortexing. The test strip was placed into the tube and lateral flow assay was performed for 5 min. The Dipsticks were photographed, and the bands were quantified using the AlphaEaseFC Software (Alphalnotech, San Leandro, Calif.).

TABLE 2
Detection probes used for HPV
RPA product detection

| | SEQ | |
|---|---|---|
| | ID | |
| Primers (5′-biotinylated) | NO: | Description |
| HPVDET1: 5′-TAATTTTAAGGAATA-3′ | 14 | Universal |
| | | Detection |
| | | probe/HPV58 |
| HPVDET2: 5′-TCGCAGTACTAATTT-3′ | 15 | HPV68 |
| HPVDET3: 5′-ACGCAGTACAAATAT-3′ | 16 | HPV16 |
| HPVDET4: 5′-TCCCAGTACCAATTT-3′ | 17 | HPV18 |
| HPVDET5: 5′-CCGTAGTACAAATAT-3′ | 18 | HPV35 |
| HPVDET6:5′-CAGAAGTACAAATTT-3′ | 19 | Universal |
| | | Detection |
| | | probe/HPV51 |
| HPVDET7: 5′-TCGCAGTACTAATAT-3′ | 20 | HPV33 |
| HPVDET8: 5′-AGATACTACTCGTAG-3′ | 21 | Universal |
| | | Detection |
| | | probe |
| HPVDET9: | 22 | Universal |
| 5′-AGATACTACTCGTAGTAC-3′ | | Detection |
| | | probe |
| | | (extended |
| | | HPVDET8) |
| HPVDET11: | 23 | Universal |
| 5′-TAATTTTAAGGAAT-3′ | | Detection |
| | | probe |
| | | (shortened |
| | | HPVDET1) |

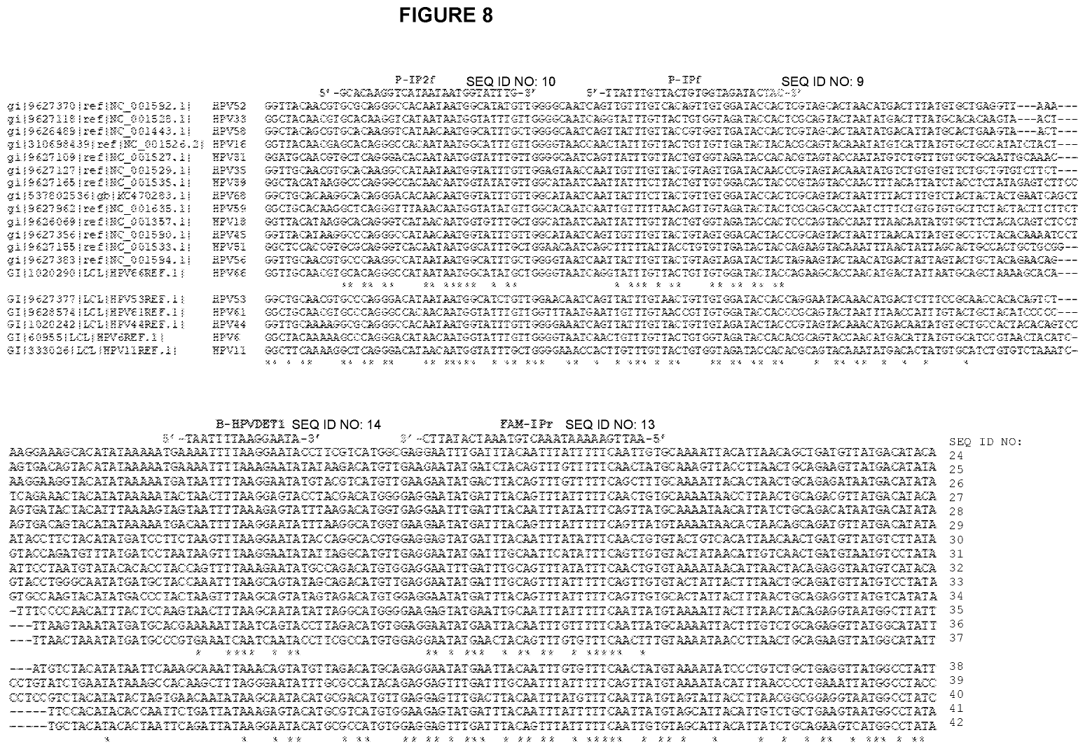

Sequence alignments: For the design of specific and consensus RPA amplification primers and universal consensus detection probes for the 14 hrHPV types relevant for cervical cancer, ClustalW sequence alignment of the 14 high-risk and 5 low-risk HPV types was preformed using a web based software (27) with the following Genbank sequence files (set forth in FIG. 8):

High-Risk HPV Types

gi|310698439|ref|NC_001526.2| Human papillomavirus type 16 (HPV16)

gi|9626069|ref|NC_001357.1| Human papillomavirus type 18 (HPV18)

gi|9627109|ref|NC_001527.1| Human papillomavirus type 31 (HPV31)

gi|9627118|ref|NC_001528.1| Human papillomavirus type 33 (HPV33)

gi|9627127|ref|NC_001529.1| Human papillomavirus type 35 (HPV35)

gi|9627165|ref|NC_001535.1| Human papillomavirus type 39 (HPV39)

gi|9627356|ref|NC_001590.1| Human papillomavirus type 45 (HPV45)

gi|9627155|ref|NC_001533.1| Human papillomavirus type 51 (HPV51)

gi|9627370|refINC_001592.1| Human papillomavirus type 52 (HPV52)

gi|9627383|ref|NC_001594.1| Human papillomavirus type 56 (HPV56)

gi|9626489|ref|NC_001443.1| Human papillomavirus type 58 (HPV58)

gi|9627962|ref|NC_001635.1| Human papillomavirus type 59 (HPV59)

gi|1020290|LCL|HPV66REF.1| Human papillomavirus type 66 (HPV66)

gi|537802536|gb|KC470283.1| Human papillomavirus type 68 (HPV68)

Low-Risk HPV Types

gi|60955|1c1|HPV6REF.1| Human papillomavirus 6 (HPV6)

gi|333026|1c1|HPV11REF.1| Human papillomavirus 11 (HPV11)

gi|1020242|1c1|HPV44REF.1| Human papillomavirus 44 (HPV44)

gi|9627377|1c1|HPV53REF.1| Human papillomavirus 53 (HPV53)

gi|9628574|1c1|HPV61REF.1| Human papillomavirus 61 (HPV61)

### Example 1—Selection of the Isothermal RPA Assay for Detection of 14 High-Risk HPV Types

Amplification and detection of all 14 hrHPV types has been previously achieved using the polymerase chain reaction (PCR) (21). Since the genomic sequence of the 14 hrHPV types with high-risk for cervical cancer is highly variable, specific primers must be used for optimal detection of each virus. For practical reasons, several universal or consensus primer pairs have been developed that target common sequences with higher similarity between the hrHPV types, such as the GP5/GP6, GPS+/GP6+, MY09/MY11, pI-1/pI-2, LIC1/LIC2-1/LIC-2-2 and PGMY09/PGMY11 primer pairs (Table 3) (26, 28-31). These consensus primers usually anneal with mismatches to their multiple targets and therefore require annealing temperatures that are below their optimal Tm (Table 3) increasing the risk of background amplifications due to primer dimers and mispriming at other targets.

TABLE 3
A selection of the most frequently
used PCR primers for HPV detection

| | Recommended | | | |
|---|---|---|---|---|
| | annealing | | Characteristics | |
| | Temperature | | (HPV target | |
| Primer pair | for PCR | | region) | References |
| MY09/MY11 | 53-55° | C. | 24 pairs of | (30, 32, 33) |
| | | | degenerate | |
| | | | primers (L1) | |
| PGMY09/PGMY11 | 53-55° | C. | Consensus | (31, 34) |
| | | | primers (L1) | |
| pI-1/pI-2 | 55° | C. | Like MY09/ | (30, 35) |
| | | | MYO9 but | |
| | | | containing | |
| | | | inosine (L1) | |
| LIC1/LIC2-1/LIC2-2 | 53° | C. | 3 consensus | (30, 36) |
| | | | primers (L1) | |
| GP5/GP6 | 45° | C. | Consensus | (30, 37) |
| | | | primers (L1) | |
| GP5+/GP6+ | 38-40° | C. | Consensus | (26, 30) |
| | | | primers (L1) | |

The PCR amplification protocols with these primer pairs usually use annealing for a very short time (seconds) at a relatively high Tm (>40-50° C.) which must be optimized to avoid background signals due to primer dimer formation and mispriming at other targets, e.g., by nested or touch-down PCR (29, 30, 38).

In contrast, for isothermal amplification methods, the primers will be present in the reaction for much longer at or close to their annealing temperature increasing the risk of primer-dimer formation, mispriming and non-specific amplification. Moreover, a much lower Tm (20-37° C.) is usually desired for point of care assays to eliminate the need for electricity and a PCR machine. Therefore, several isothermal methods for amplification of DNA such as rolling circle amplification (RCA) (39-41), helicase-dependent amplification (HDA) (42), loop-mediated amplification (LAMP) (22, 43), or recombinase polymerase amplification (RPA) (24) have been evaluated and tested for their ability to amplify hrHPV.

### Example 2—Detection Limit of RPA with Specific Primers for hrHPV

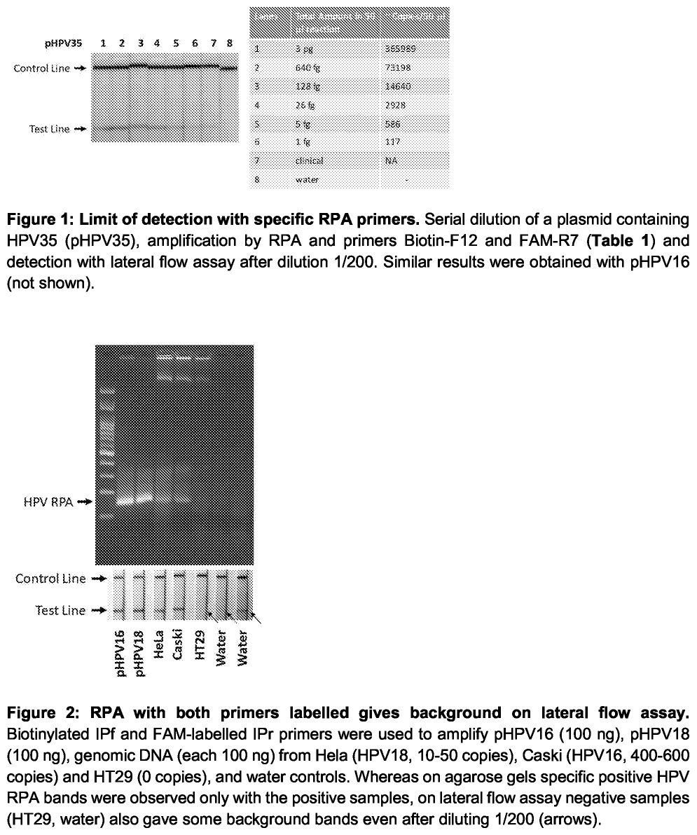

Among the 12 primer sets tested experimentally, two specific primer pairs for hrHPV16 (F3/R2, SEQ ID NOs: 2 and 4, respectively) and hrHPV35 (F12/R7, SEQ ID NOs: 5 and 6, respectively) provided in Table 1 were selected to evaluate whether RPA is efficient enough to detect specific hrHPV types in plasmids, genomic DNA from cell lines and clinical samples. To this end, a plasmid containing HPV35 (pHPV35) was serially diluted and RPA performed using the F12/R7 primer pair (SEQ ID NOs: 5 and 6, respectively) labelled at the 5′-ends with biotin or 6-carboxyfluorescein (FAM), respectively, and the labelled double-stranded RPA reaction product detected with lateral flow assay (FIG. 1). Careful dilution of the RPA reaction product (between 1/200 to 1/500) was necessary before loading on the paper strips since otherwise non-specific signals were observed that were due to the generation of labelled products by primer dimers and non-specific background amplification (not shown). Using the specific labelled primer pair (F3/R2, SEQ ID NOs: 3 and 4)), similar results were obtained with pHPV16 (not shown). These results indicated that by using specific and optimized primer pairs, RPA may be a suitable technique to detect low copies numbers (100-200 copies) of hrHPV as present in clinical samples. However, to detect all 14 hrHPV types using this strategy, 28 primers would be required, which is too complex and not practical for a PoC test. Therefore, as described in the following Example, universal consensus primers were designed and tested for their ability to amplify all 14 hrHPV types using the isothermal RPA technique.

### Example 3—Development of Universal Consensus Primers for Detection of High-Risk HPV by RPA

For RPA it is recommended to use primers that are longer than conventional PCR primers, e.g., primers of 30-35 nucleotides in length (TwistDX). Therefore, using ClustalW sequence alignment of the 14 hrHPV types (FIG. 8), several extended versions of the GP5+/GP6+ PCR primer pair were designed and tested in RPA reactions with plasmids and genomic DNA from cell lines (Table 1). Amongst the tested primer pairs, one set (exGP5+/exGP6+, SEQ ID NOs: 7 and 8, respectively) could amplify all 14 hrHPV types from plasmids and hrHPV16 and hrHPV18 from cell lines with reasonable efficiency but generally less efficiently when compared to the above described specific primer pairs (F3/R2 (SEQ ID NOs: 3 and 4), F12/R7 (SEQ ID NOs: 5 and 6) and with more background noise (data not shown). Since it was also reported that shorter primer pairs can work for RPA, the original GP5+/GP6+ primer pair (SEQ ID NOs: 1 and 2) used for PCR was also tested, because it had a relatively low annealing temperature of 38-40° C. making it possibly suitable for RPA. Interestingly, the original GP5+/GP6+primer pair (SEQ ID NOs: 1 and 2) could also amplify all 14 hrHPV types, but again it gave a relatively high background noise (data not shown).

Several variants of these primers were tested with no change; however, using combinations of primers of various length derived from the above primer pairs gave some improvements. A novel primer pair was designed again based on the ClustalW sequence alignment of the 14 hrHPV types using RPA that was intermediate in length (IPf/IPr, SEQ ID NOs: 9 and 11, respectively). Interestingly, this primer pair worked efficiently with all 14 hrHPV types, genomic DNA from cell lines and clinical samples and gave less background due to non-specific priming on agarose gel (FIG. 2).

However, when tested on lateral flow assay these labelled IPf/IPr primers (SEQ ID NOs: 9 and 11) still gave some background (FIG. 2). Inclusion of the universal base inosine into IPf/IPr, generating IPfino/IPrino (SEQ ID NOs: 12 and 13, respectively), did not improve amplification efficiency when using the same amplification conditions (data not shown). The IPf/IPr consensus primer pair (SEQ ID NOs: 9 and 11) was further used in the following experiments with the goal to develop a low-cost point of care test with paper-based lateral flow assay.

### Example 4—Detection of RPA Products by Paper-Based Lateral Flow Assay

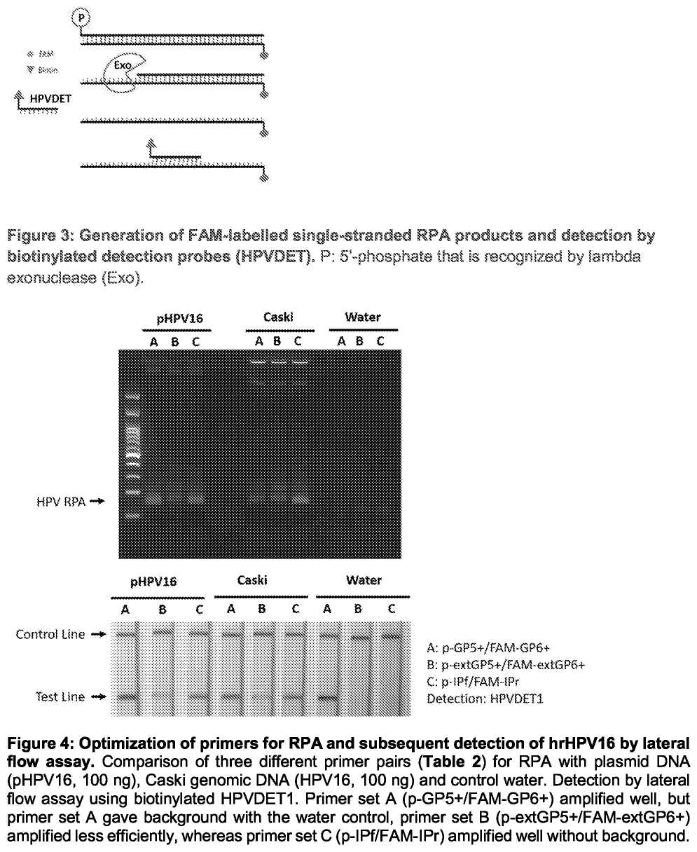

The RPA product is double-stranded and in lateral flow assays, the use of a biotinylated forward and a fluorescent reverse primer for amplification did result in background due to primer dimers and unspecific amplification by RPA (FIG. 2) (and also with PCR, not shown). Therefore, a method for converting double-stranded products to single-stranded products used for sequencing and microarrays was adopted and developed for the detection of RPA products (44, 45). In this method, one primer is labelled with 6-carboxyfluorescein (FAM) and the other is phosphorylated at the 5′-end. Lambda exonuclease preferentially digests double-stranded DNA from the 5′-phosphorylated end (46, 47), whereas fluorescent-labelled ends (Cy3, Cy5, FAM) are protected (FIG. 3) (48-51). To increase amplification, several phosphorylated primers may be used in tandem, since they are not labelled by themselves and therefore will give no background. To detect the single-stranded FAM-labelled RPA product, biotin-labelled detection probes were generated (Table 2 above) that anneal to the single-stranded RPA product (these biotin labelled detection probes are protected against digestion by residual lambda exonuclease activity (48)). When compared to the approach using two labelled primers (FIG. 2), this method is expected to give less background since only one labelled primer (FAM) is present during the RPA reaction, and detection occurs afterwards using biotinylated universal consensus detection probes hybridizing to an internal sequence that will only be present after successful amplification of hrHPV. Indeed, when different primer sets were compared using this detection method, the p-IPf/FAM-IPr primer pair (SEQ ID NO: 9 and SEQ ID NO: 11 (5′-phosphorylated and labeled at the 5′ end with 6-carboxyfluorescein (FAM), respectively) worked best with HPV16 in plasmid and genomic DNA and gave less background (FIG. 4).

### Example 5—Detection of High-Risk HPV with Universal Detection Probes for Lateral Flow Assay

For detection of the 14 hrHPV RPA products, 14 detection probes would be required to achieve detection without mismatch. In fact, specific detection probes selected from a pool of random oligonucleotides after several rounds of hybridization were reported to detect their hrHPV type at ambient temperature (49, 52). Therefore, several biotinylated specific detection probes were designed that specifically can detect their hrHPV type (Table 2). Efficient detection of individual hrHPV types was achieved by using these specific detection probes (not shown).

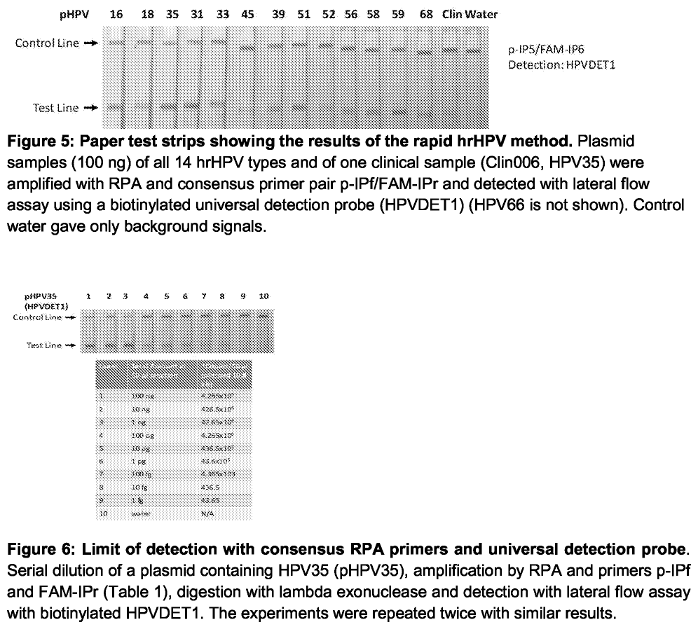

Towards the development of an universal detection probe able to detect all 14 hrHPV types, the ClustalW sequence alignment of the sequences of the 14 hrHPV types was used to determine regions of high homology within the sequence of the amplified RPA fragments (FIG. 8). Several universal detection probes that hybridize to common sequences were tested for their ability to detect all hrHPV types (Table 2). However, using conventional assay conditions provided by the manufacturer (Milenia Biotec), these detection probes were often not able to detect the ssDNA FAM-labelled RPA fragments (data not shown), most likely because at room temperature annealing to the target did not occur in the presence of mismatches. In fact, the calculated Tm of non-mismatched annealing was around 34° C. (as calculated with Integrated DNA technology software), not suitable for annealing with mismatches to their target sequence. Therefore, the Tm was lowered by adding Tm depressors (NaCl (420 mM) and MgCl₂ (83 mM)) (53) to the lateral flow assay buffer. Using these conditions, the universal detection probe (HPVDET1, SEQ ID NO: 14) was able to detect all 14 hrHPV types amplified from plasmid templates and HPV35 from a clinical sample using lateral flow assay (FIG. 5). HPVDET1 (SEQ ID NO: 14) has a higher homology to the 14 hrHPV types (8 of 15 bases in common, except HPV66) when compared to 5 low-risk HPV types (4 of 15 bases in common) and therefore is expected to preferentially detect the 14 hrHPV types (data not shown).

### Example 6—Detection Limit of Universal Consensus Primer with HPV35 Plasmid DNA and Lateral Flow Assay with Universal Detection Probe

To evaluate whether RPA with the optimized consensus amplification and detection probes is efficient enough to detect specific hrHPV types, a plasmid containing HPV35 (pHPV35) was serially diluted and RPA performed using consensus p-IPf/FAM-IPr primers (SEQ ID NO: 9 and SEQ ID NO: 11 (5′-phosphorylated or labeled at the 5′ end with 6-carboxyfluorescein (FAM), respectively), and detection with the biotinylated probe HPVDET1 (SEQ ID NO: 14) (FIG. 6). A detection limit of 10 fg or about 450 copies hrHPV35 was achieved.

### Example 7—Improving the Detection of Clinical Samples with Alternative Primers and Detection Probes

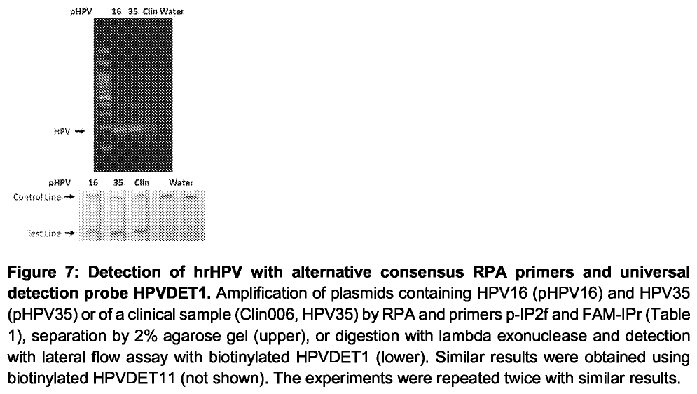

Alternative primers (IP2f, SEQ ID NO: 10) and detection probes (HPVDET11, SEQ ID NO: 23) were designed based on the ClustalW sequence alignment of the sequences of the 14 hrHPV types (27) (FIG. 8). Interestingly, the IP2f/IPr pimer pair (SEQ ID NOs: 10 and 11) could amplify HPV16 from Caski cells and clinical sample (clinical006, HPV35) with high efficiency (FIG. 7) and was able to amplify all 14 hrHPV types, with a detection limit for hrHPV35 of about 60 copies. Based on the sequence alignment, HPVDET1 (SEQ ID NO: 14) and HPVDET11 (SEQ ID NO: 23) are more likely to detect hrHPV compared to low-risk HPV (FIG. 8).

Discussion

Over 95% of cervical cancers are caused by hrHPV (12). Thus, primary hrHPV DNA testing has been integrated into most routine cervical cancer screenings in clinical settings. Studies spanning a broad range of geographic, ethnic, and socioeconomic groups comparing hrHPV DNA testing with Pap smear screening have found that hrHPV DNA testing has a higher sensitivity and negative predictive value (54). Additionally, hrHPV testing does not always require a clinician, given the advent of hrHPV self-sampling populations (10, 55, 56). hrHPV self-sampling enables women to test themselves for hrHPV outside a clinical setting and has consistently been shown to be equivalent to physician-collected specimens for hrHPV detection. Moreover, rapid hrHPV DNA testing will eliminate the need for follow-up consultation, breaching a significant barrier to effective treatment.

Currently over 30 HPV assays are commercially available, but only 7 are FDA approved including the Gen-Probe APTIMA HPV assay, the Hologic Cervista HPV HR test, the Roche Cobas HPV test, the QIAGEN Hybrid Capture 2 HPV DNA test (HC2) and most recently the BD Oncoclarity test (9). These tests are used to identify hrHPV DNA and to allow for risk stratification and evaluation of subsequent management strategies. The high cost and need for laboratory expertise have dampened the worldwide use of hrHPV DNA testing. Moreover, the time required from sampling to result increases the number of women lost to follow-up. Perhaps most importantly, current commercially available assays necessitate laboratory-trained personnel to perform them, which can be challenging in low resource settings. Therefore, a simple low-cost hrHPV Point of Care test (PoCT) that allows self-sampling and rapid detection of the 14 hrHPV types is of high value.

The rapid hrHPV detection method described herein is paper-based, relatively cheap to produce, and easy to use facilitating self-sampling for use in on site assays. Such paper platforms provide advantages in both resource-poor settings and/or environments with a lack of infrastructure and trained personnel needed for laboratory testing. The method was initially developed for two specific hrHPV types, HPV16 and HPV35, and it was sensitive enough sto detect hrHPV in clinical samples. However, since there are 14 hrHPV types, it would require 28 primer pairs and 14 detection probes and 14 reactions what is not practical in a point of care setting. Therefore, “universal” consensus primer pairs and “universal” consensus detection probes were developed that were capable of detecting all 14 hrHPV types with only 2 primers and 1 detection probe. These primers were sensitive enough to detect HPV35 in clinical samples within short time.

References:

1. zur Hausen H. (2009) Papillomaviruses in the causation of human cancers—a brief historical account. Virology 384, 260-265.
2. Faridi R., Zahra A., Khan K., and Idrees M. (2011) Oncogenic potential of Human Papillomavirus (HPV) and its relation with cervical cancer. Virol J 8, 269.
3. Munoz N., Bosch F. X., de Sanjose S., Herrero R., Castellsague X., Shah K. V., Snijders P. J., Meijer C. J., and International Agency for Research on Cancer Multicenter Cervical Cancer Study G. (2003) Epidemiologic classification of human papillomavirus types associated with cervical cancer. The New England journal of medicine 348, 518-527.
4. Schiffman M., Herrero R., Desalle R., Hildesheim A., Wacholder S., Rodriguez A. C., Bratti M. C., Sherman M. E., Morales J., Guillen D., Alfaro M., Hutchinson M., Wright T. C., Solomon D., Chen Z., Schussler J., Castle P. E., and Burk R. D. (2005) The carcinogenicity of human papillomavirus types reflects viral evolution. Virology 337, 76-84.
5. Smith J.S., Lindsay L., Hoots B., Keys J., Franceschi S., Winer R., and Clifford G. M. (2007) Human papillomavirus type distribution in invasive cervical cancer and high-grade cervical lesions: a meta-analysis update. Int J Cancer 121, 621-632.
6. Walboomers J. M., Jacobs M. V., Manos M. M., Bosch F. X., Kummer J. A., Shah K. V., Snijders P. J., Peto J., Meijer C. J., and Munoz N. (1999) Human papillomavirus is a necessary cause of invasive cervical cancer worldwide. J Pathol 189, 12-19.
7. Garland S. M., Kjaer S. K., Munoz N., Block S. L., Brown D. R., DiNubile M. J., Lindsay B. R., Kuter B. J., Perez G., Dominiak-Felden G., Saah A. J., Drury R., Das R., and Velicer C. (2016) Impact and Effectiveness of the Quadrivalent Human Papillomavirus Vaccine: A Systematic Review of 10 Years of Real-world Experience. Clin Infect Dis 63, 519-527.
8. Van Doorslaer K. (2013) Evolution of the papillomaviridae. Virology 445, 11-20.
9. Cubie H. A. and Cuschieri K. (2013) Understanding HPV tests and their appropriate applications. Cytopathology 24, 289-308.
10. Kobetz E., Seay J., Koru-Sengul T., Bispo J. B., Trevil D., Gonzalez M., Brickman A., and Carrasquillo O. (2018) A randomized trial of mailed HPV self-sampling for cervical cancer screening among ethnic minority women in South Florida. Cancer Causes Control 29, 793-801.
11. Abreu A. L., Souza R. P., Gimenes F., and Consolaro M. E. (2012) A review of methods for detect human Papillomavirus infection. Virol J 9, 262.
12. Kroupis C. and Vourlidis N. (2011) Human papilloma virus (HPV) molecular diagnostics. Clinical chemistry and laboratory medicine 49, 1783-1799.
13. Chang C. C., Chen C. C., Wei S. C., Lu H. H., Liang Y. H., and Lin C. W. (2012) Diagnostic devices for isothermal nucleic acid amplification. Sensors (Basel) 12, 8319-8337.
14. Craw P. and Balachandran W. (2012) Isothermal nucleic acid amplification technologies for point-of-care diagnostics: a critical review. Lab on a chip 12, 2469-2486.
15. Rodriguez N. M., Wong W. S., Liu L., Dewar R., and Klapperich C. M. (2016) A fully integrated paperfluidic molecular diagnostic chip for the extraction, amplification, and detection of nucleic acids from clinical samples. Lab on a chip 16, 753-763.
16. Zanoli L. M. and Spoto G. (2013) Isothermal amplification methods for the detection of nucleic acids in microfluidic devices. Biosensors (Basel) 3, 18-43.
17. Troeger V., Niemann K., Gaertig C., and Kuhlmeier D. (2015) Isothermal Amplification and Quantification of Nucleic Acids and its Use in Microsystems. J Nanomeicine & Nanotechnology 6.
18. Yan L., Zhou J., Zheng Y., Gamson A. S., Roembke B. T., Nakayama S., and Sintim H. O. (2014) Isothermal amplified detection of DNA and RNA. Mol Biosyst 10, 970-1003.
19. Sharma N., Hoshika S., Hutter D., Bradley K. M., and Benner S. A. (2014) Recombinase-based isothermal amplification of nucleic acids with self-avoiding molecular recognition systems (SAMRS). Chembiochem: a European journal of chemical biology 15, 2268-2274.
20. Daher R. K., Stewart G., Boissinot M., and Bergeron M. G. (2016) Recombinase Polymerase Amplification for Diagnostic Applications. Clinical chemistry 62, 947-958.
21. Sun K., Xing W., Yu X., Fu W., Wang Y., Zou M., Luo Z., and Xu D. (2016) Recombinase polymerase amplification combined with a lateral flow dipstick for rapid and visual detection of Schistosoma japonicum. Parasit Vectors 9, 476.
22. Song J., Liu C., Mauk M. G., Rankin S. C., Lok J. B., Greenberg R. M., and Bau H. H. (2017) Two-Stage Isothermal Enzymatic Amplification for Concurrent Multiplex Molecular Detection. Clinical chemistry.
23. Ereku L. T., Mackay R. E., Craw P., Naveenathayalan A., Stead T., Branavan M., and Balachandran W. (2018) RPA using a multiplexed cartridge for low cost point of care diagnostics in the field. Analytical biochemistry 547, 84-88.

### 24. Ma B., Fang J., Wang Y., He H., Dai M., Lin W., Su W., and Zhang M. (2017) Isothermal Method of a Recombinase Polymerase Amplification Assay for the Detection of Most Common High-Risk Human Papillomavirus Type 16 and Type 18 DNA. Clin Lab 63, 27-38.

25. Daher R. K., Stewart G., Boissinot M., Boudreau D. K., and Bergeron M. G. (2015) Influence of sequence mismatches on the specificity of recombinase polymerase amplification technology. Mol Cell Probes 29, 116-121.
26. de Roda Husman A. M., Walboomers J. M., van den Brule A. J., Meijer C. J., and Snijders P. J. (1995) The use of general primers GP5 and GP6 elongated at their 3′ ends with adjacent highly conserved sequences improves human papillomavirus detection by PCR. The Journal of general virology 76 (Pt 4), 1057-1062.
27. Chojnacki S., Cowley A., Lee J., Foix A., and Lopez R. (2017) Programmatic access to bioinformatics tools from EMBL-EBI update: 2017. Nucleic acids research 45, W550-W553.
28. Qu W., Jiang G., Cruz Y., Chang C. J., Ho G. Y., Klein R. S., and Burk R. D. (1997) PCR detection of human papillomavirus: comparison between MY09/MY11 and GP5+/GP6+ primer systems. Journal of clinical microbiology 35, 1304-1310.
29. Evans M. F., Adamson C. S., Simmons-Arnold L., and Cooper K. (2005) Touchdown General Primer (GP5+/GP6+) PCR and optimized sample DNA concentration support the sensitive detection of human papillomavirus. BMC clinical pathology 5, 10.
30. Husnjak K., Grce M., Magdic L., and Pavelic K. (2000) Comparison of five different polymerase chain reaction methods for detection of human papillomavirus in cervical cell specimens. Journal of virological methods 88, 125-134.
31. Erhart S. M., Rivero E. R., Bazzo M. L., and Onofre A. S. (2016) Comparative evaluation of the GP5+/6+, MY09/11 and PGMY09/11 primer sets for HPV detection by PCR in oral squamous cell carcinomas. Experimental and molecular pathology 100, 13-16.
32. Manos M. M., Ting Y., Wright D. K., Lewis A. I., Broker T. R., and Wolinsky S. M. (1989) The use of polymerase chain reaction amplification for the detection of genital human papillomaviruses. Cancer cells 7, 209-214.
33. Sahiner F., Kubar A., Gumral R., Ardic M., Yigit N., Sener K., Dede M., and Yapar M. (2014) Efficiency of MY09/11 consensus PCR in the detection of multiple HPV infections. Diagnostic microbiology and infectious disease 80, 43-49.
34. Gravitt P. E., Peyton C. L., Alessi T. Q., Wheeler C. M., Coutlee F., Hildesheim A., Schiffman M. H., Scott D. R., and Apple R. J. (2000) Improved amplification of genital human papillomaviruses. Journal of clinical microbiology 38, 357-361.
35. Novelli G., Gennarelli M., De Santis L., Angeloni P., and Dallapiccola B. (1992) Inosine-containing primers in human papillomavirus detection by polymerase chain reaction. Biomed Pharmacother 46, 167-169.
36. Yoshikawa H., Kawana T., Kitagawa K., Mizuno M., Yoshikura H., and Iwamoto A. (1990) Amplification and typing of multiple cervical cancer-associated human papillomavirus DNAs using a single pair of primers. Int J Cancer 45, 990-992.
37. Snijders P. J., van den Brule A. J., Schrijnemakers H. F., Snow G., Meijer C. J., and Walboomers J. M. (1990) The use of general primers in the polymerase chain reaction permits the detection of a broad spectrum of human papillomavirus genotypes. The Journal of general virology 71 (Pt 1), 173-181.
38. Fuessel Haws A. L., He Q., Rady P. L., Zhang L., Grady J., Hughes T. K., Stisser K., Konig R., and Tyring S. K. (2004) Nested PCR with the PGMY09/11 and GP5(+)/6(+) primer sets improves detection of HPV DNA in cervical samples. Journal of virological methods 122, 87-93.
39. Xu X., Zhang B., Gan P., Wu J., Dai W., Zhang L., and Wang J. (2017) On-nylon membrane detection of nucleic acid molecules by rolling circle amplification. Analytical biochemistry 533, 26-33.
40. Lu W., Yuan Q., Yang Z., and Yao B. (2017) Self-primed isothermal amplification for genomic DNA detection of human papillomavirus. Biosensors & bioelectronics 90, 258-263.

### 41. Zingg J. -M. and Daunert S. (2018) Trinucleotide Rolling Circle Amplification: A Novel Method for the Detection of RNA and DNA. Methods and Protocols 1, 15.

42. Barbieri D., Venturoli S., Rosl F., and Rincon-Orozco B. (2014) Detection of high-risk human papillomavirus type 16 and 18 using isothermal helicase-dependent amplification. Diagnostic microbiology and infectious disease 79, 178-182.
43. Lin J., Ma B., Fang J., Wang Y., He H., Lin W., Su W., and Zhang M. (2017) Colorimetric Detection of 23 Human Papillomavirus Genotypes by Loop-Mediated Isothermal Amplification. Clin Lab 63, 495-505.
44. Franke-Whittle I. H., Klammer S. H., and Insam H. (2005) Design and application of an oligonucleotide microarray for the investigation of compost microbial communities. Journal of microbiological methods 62, 37-56.
45. Higuchi R. G. and Ochman H. (1989) Production of single-stranded DNA templates by exonuclease digestion following the polymerase chain reaction. Nucleic acids research 17, 5865.
46. Little J. W. (1967) An exonuclease induced by bacteriophage lambda. II. Nature of the enzymatic reaction. The Journal of biological chemistry 242, 679-686.
47. Little J. W., Lehman I. R., and Kaiser A. D. (1967) An exonuclease induced by bacteriophage lambda. I. Preparation of the crystalline enzyme. The Journal of biological chemistry 242, 672-678.
48. Murgha Y. E., Rouillard J. M., and Gulari E. (2014) Methods for the preparation of large quantities of complex single-stranded oligonucleotide libraries. PLoS One 9, e94752.
49. Brukner I., Krajinovic M., Dascal A., and Labuda D. (2007) A protocol for the in vitro selection of specific oligonucleotide probes for high-resolution DNA typing. Nature protocols 2, 2807-2814.
50. Brinker A., Schulze H., Bachmann T., and Moller R. (2010) Lambda exonuclease pre-treatment for improved DNA-chip performance depends on the relative probe-target position. Biosensors & bioelectronics 26, 898-902.
51. Boissinot K., Huletsky A., Peytavi R., Turcotte S., Veillette V., Boissinot M., Picard F. J., m A. E., and Bergeron M. G. (2007) Rapid exonuclease digestion of PCR-amplified targets for improved microarray hybridization. Clinical chemistry 53, 2020-2023.
52. Brukner I., El-Ramahi R., Sawicki J., Gorska-Flipot I., Krajinovic M., and Labuda D. (2007) Hybridization assay performed at ambient temperature for typing high-risk human papillomaviruses. Journal of clinical virology: the official publication of the Pan American Society for Clinical Virology 39, 113-118.
53. Doyle S. R., Chan C. K., and Grant W. N. (2011) Enhanced annealing of mismatched oligonucleotides using a novel melting curve assay allows efficient in vitro discrimination and restriction of a single nucleotide polymorphism. BMC biotechnology 11, 83.
54. Mayrand M. H., Duarte-Franco E., Rodrigues I., Walter S. D., Hanley J., Ferenczy A., Ratnam S., Coutlee F., Franco E. L., and Canadian Cervical Cancer Screening Trial Study G. (2007) Human papillomavirus DNA versus Papanicolaou screening tests for cervical cancer. The New England journal of medicine 357, 1579-1588.
55. Kobetz E., Seay J., Amofah A., Pierre L., Bispo J. B., Trevil D., Gonzalez M., Poitevien M., Koru-Sengul T., and Carrasquillo O. (2017) Mailed HPV self-sampling for cervical cancer screening among underserved minority women: study protocol for a randomized controlled trial. Trials 18, 19.
56. Carrasquillo O., Seay J., Amofah A., Pierre L., Alonzo Y., McCann S., Gonzalez M., Trevil D., Koru-Sengul T., and Kobetz E. (2018) HPV Self-Sampling for Cervical Cancer Screening Among Ethnic Minority Women in South Florida: a Randomized Trial. J Gen Intern Med 33, 1077-1083.
57. Rodriguez N. M., Linnes J. C., Fan A., Ellenson C. K., Pollock N. R., and Klapperich C. M. (2015) Paper-Based RNA Extraction, in Situ Isothermal Amplification, and Lateral Flow Detection for Low-Cost, Rapid Diagnosis of Influenza A (H1N1) from Clinical Specimens. Analytical chemistry 87, 7872-7879.
58. Butzler M. A., Reed J. L., and McFall S. M. (2017) A simple and rapid DNA extraction method for Chlamydia trachomatis detection from urogenital swabs. Diagnostic microbiology and infectious disease 89, 182-184.
59. McFall S. M., Wagner R. L., Jangam S. R., Yamada D. H., Hardie D., and Kelso D. M. (2015) A simple and rapid DNA extraction method from whole blood for highly sensitive detection and quantitation of HIV-1 proviral DNA by real-time PCR. Journal of virological methods 214, 37-42.
60. Jangam S. R., Yamada D. H., McFall S. M., and Kelso D. M. (2009) Rapid, point-of-care extraction of human immunodeficiency virus type 1 proviral DNA from whole blood for detection by real-time PCR. Journal of clinical microbiology 47, 2363-2368.
61. Bordelon H., Russ P. K., Wright D. W., and Haselton F. R. (2013) A magnetic bead-based method for concentrating DNA from human urine for downstream detection. PLoS One 8, e68369.

What is claimed is:

1. A kit for the detection of high risk human papilloma virus (hrHPV) polynucleotides in a biological sample comprising
(a) a primer pair; and
(b) a detection probe;
wherein (a) and (b) are capable of detecting each of the hrHPV polynucleotides, if present, in the sample by recombinase polymerase amplification (RPA).

2. The kit of claim 1, further comprising a running buffer.

3. The kit of claim 1 or claim 2, further comprising a test strip.

4. The kit of any one of claims 1-3, wherein the primer pair comprises the nucleotide sequence set forth in SEQ ID NOs: 9 and 11.

5. The kit of any one of claims 1-3, wherein the primer pair comprises the nucleotide sequence set forth in SEQ ID NOs: 10 and 11.

6. The kit of any one of claims 1-3, wherein the primer pair comprises the nucleotide sequence set forth in SEQ ID NOs: 12 and 13.

7. The kit of any one of claims 1-3, wherein the primer pair comprises a first primer that is phosphorylated at the 5′-end and a second primer is labeled with 6-carboxyfluorescein (FAM).

8. The kit of claim 4, wherein the nucleotide sequence set forth in SEQ ID NO: 9 is phosphorylated and the nucleotide sequence set forth in SEQ ID NO: 11 is labeled with 6-carboxyfluorescein (FAM).

9. The kit of any one of claims 1-6, wherein the detection probe comprises a nucleotide sequence set forth in SEQ ID NO: 14 (HPVDET1) or SEQ ID NO: 23 (HPVDET11).

10. The kit of claim 9, wherein the detection probe comprises the nucleotide sequence set forth in SEQ ID NO: 14 (HPVDET1).

11. The kit of any one of claims 1-8, wherein the detection probe is biotinylated.

12. The kit of any one of claims 1-8, wherein the hrHPV is HPV16, HPV18, HPV31, HPV33, HPV35, HPV39, HPV45, HPV51, HPV52, HPV56, HPV58, HPV59, HPV66 or HPV68.

13. The kit of any one of claims 1-10, wherein the primer pair is capable of amplifying each of HPV16, HPV18, HPV35, HPV31, HPV33, HPV35, HPV39, HPV45, HPV51, HPV52, HPV56, HPV58, HPV59, HPV66 and HPV68 in a biological sample.

14. The kit of any one of claims 2-13, wherein the running buffer comprises magnesium chloride and sodium chloride.

15. The kit of any one of claims 2-14, wherein the running buffer comprises about 420 mM sodium chloride, and about 83 mM magnesium chloride, pH 6.5-8.5.

16. The kit of any one of claims 3-15, wherein the test strip comprises filter paper.

17. The kit of any one of claims 3-16, wherein the test strip comprises chitosan.

18. A method for detecting high risk human papilloma virus (hrHPV) polynucleotides in a biological sample comprising:
(a) an amplifying step comprising adding the biological sample to a vessel containing a primer pair that is capable of amplifying each of the hrHPV polynucleotides, if present, in the biological sample,
(b) digesting amplified hrHPV polynucleotides in the vessel into a single-stranded amplified product;
(c) combining the single-stranded amplified product with a running buffer comprising a detection probe that is capable of detecting the single-stranded amplified product to form a mixture, and incubating the mixture for a period of time in the vessel; and,
(d) a detecting step comprising wicking the mixture into a test strip and visually detecting the detection probe on the test strip.

19. The method of claim 18, wherein the digesting step comprises adding an exonuclease to the vessel before the detecting step.

20. The method of claim 19, wherein the exonuclease is lambda exonuclease.

21. The method of claim 18, wherein the amplifying step does not comprise incubating the mixture at a temperature greater than about 37° C.

22. The method of any one of claims 18-21, wherein the amplifying step comprises incubating the mixture at a temperature between about 20° C. and about 37° C.

23. The method of any one of claims 18-21, wherein the amplifying step does not comprise incubating the mixture at a temperature greater than about 30° C.

24. The method of any of claims 18-23, wherein the amplifying step comprises incubating the mixture for about 10 minutes to about 2 hours.

25. The method of any of claims 18-24, wherein the amplifying step comprises incubating the mixture for about 10 to about 20 minutes.

26. The method of any one of claims 18-25, wherein the amplifying step and/or the detecting step is performed without additional instrumentation.

27. The method of any one of claims 18-26, wherein the primer pair comprises the nucleotide sequences set forth in SEQ ID NOs: 9 and 11.

28. The method of any one of claims 18-27, wherein the primer pair comprises the nucleotide sequence set forth in SEQ ID NOs: 10 and 11.

29. The method of any one of claims 18-28, wherein the primer pair comprises the nucleotide sequence set forth in SEQ ID NOs: 12 and 13.

30. The method of any one of claims 18-29, wherein the primer pair comprises a first primer is phosphorylated at the 5′-end and a second primer is labeled with 6-carboxyfluorescein (FAM).

31. The method of claim 30, wherein the nucleotide sequence set forth in SEQ ID NO: 9 is phosphorylated and the nucleotide sequence set forth in SEQ ID NO: 11 is labeled with 6-carboxyfluorescein (FAM).

32. The method of any one of claims 18-31, wherein the detection probe is SEQ ID NO: 14 (HPVDET1) of SEQ ID NO: 23 (HPVDET11).

33. The method of any one of claims 18-32, wherein the detection probe is SEQ ID NO: 14 (HPVDET11).

34. The method of any one of claims 18-33, wherein the detection probe is biotinylated.

35. The method of any one of claims 18-34, wherein the hrHPV is HPV16, HPV18, HPV31, HPV33, HPV35, HPV39, HPV45, HPV51, HPV52, HPV56, HPV58, HPV59, HPV66 or HPV68.

36. A kit comprising
(a) a consensus primer pair capable of amplifying multiple species of high risk human papilloma virus (hrHPV) in a biological sample;
(b) a detection probe;
(c) a test strip.


SEQUENCE LISTING (42 sequences)

SEQ ID NO: 1 - DNA, 23 bp, Artificial Sequence
  Synthetic Polynucleotide
tttgttactgtggtagatactac

SEQ ID NO: 2 - DNA, 25 bp, Artificial Sequence
  Synthetic Polynucleotide
gaaaaataaactgtaaatcatattc

SEQ ID NO: 3 - DNA, 30 bp, Artificial Sequence
  Synthetic Polynucleotide
  misc_feature (1)..(1): FAM
aaacaactatacatgatataatattagaat

SEQ ID NO: 4 - DNA, 30 bp, Artificial Sequence
  Synthetic Polynucleotide
  misc_feature (4)..(4): Biotin
aacaagacatacatcgaccggtccaccgac

SEQ ID NO: 5 - DNA, 30 bp, Artificial Sequence
  Synthetic Polynucleotide
  misc_feature (1)..(1): FAM
aaaagcaaagacatttagaagaaaaaaaac

SEQ ID NO: 6 - DNA, 30 bp, Artificial Sequence
  Synthetic Polynucleotide
  misc_feature (1)..(1): Biotin
gctggaccgtcaatggtatcttcctcctcc

SEQ ID NO: 7 - DNA, 29 bp, Artificial Sequence
  Synthetic Polynucleotide
cagttatttgttactgtggtagatactac

SEQ ID NO: 8 - DNA, 32 bp, Artificial Sequence
  Synthetic Polynucleotide
cacaattgaaaaataaactgtaaatcatattc

SEQ ID NO: 9 - DNA, 26 bp, Artificial Sequence
  Synthetic Polynucleotide
ttatttgttactgtggtagatactac

SEQ ID NO: 10 - DNA, 26 bp, Artificial Sequence
  Synthetic Polynucleotide
gcacaaggtcataataatggtatttg

SEQ ID NO: 11 - DNA, 29 bp, Artificial Sequence
  Synthetic Polynucleotide
aattgaaaaataaactgtaaatcatattc

SEQ ID NO: 12 - DNA, 25 bp, Artificial Sequence
  Synthetic Polynucleotide
ttatttgttactgtggtagatacac

SEQ ID NO: 13 - DNA, 28 bp, Artificial Sequence
  Synthetic Polynucleotide
aattgaaaaataaactgtaaatcaattc

SEQ ID NO: 14 - DNA, 15 bp, Artificial Sequence
  Synthetic Polynucleotide
taattttaaggaata

SEQ ID NO: 15 - DNA, 15 bp, Artificial Sequence
  Synthetic Polynucleotide
tcgcagtactaattt

SEQ ID NO: 16 - DNA, 15 bp, Artificial Sequence
  Synthetic Polynucleotide
acgcagtacaaatat

SEQ ID NO: 17 - DNA, 15 bp, Artificial Sequence
  Synthetic Polynucleotide
tcccagtaccaattt

SEQ ID NO: 18 - DNA, 15 bp, Artificial Sequence
  Synthetic Polynucleotide
ccgtagtacaaatat

SEQ ID NO: 19 - DNA, 15 bp, Artificial Sequence
  Synthetic Polynucleotide
cagaagtacaaattt

SEQ ID NO: 20 - DNA, 15 bp, Artificial Sequence
  Synthetic Polynucleotide
tcgcagtactaatat

SEQ ID NO: 21 - DNA, 15 bp, Artificial Sequence
  Synthetic Polynucleotide
agatactactcgtag

SEQ ID NO: 22 - DNA, 18 bp, Artificial Sequence
  Synthetic Polynucleotide
agatactactcgtagtac

SEQ ID NO: 23 - DNA, 14 bp, Artificial Sequence
  Synthetic Polynucleotide
taattttaaggaat

SEQ ID NO: 24 - DNA, 234 bp, Homo Sapiens
ggttacaacgtgcgcagggccacaataatggcatatgttggggcaatcagttgtttgtcacagttgtggataccactcgtagcactaacatgactttatgtgctgaggttaaaaaggaaagcacatataaaaatgaaaattttaaggaataccttcgtcatggcgaggaatttgatttacaatttatttttcaattgtgcaaaattacattaacagctgatgttatgacataca

SEQ ID NO: 25 - DNA, 234 bp, Homo Sapiens
ggctacaacgtgcacaaggtcataataatggtatttgttggggcaatcaggtatttgttactgtggtagataccactcgcagtactaatatgactttatgcacacaagtaactagtgacagtacatataaaaatgaaaattttaaagaatatataagacatgttgaagaatatgatctacagtttgtttttcaactatgcaaagttaccttaactgcagaagttatgacatata

SEQ ID NO: 26 - DNA, 234 bp, Homo Sapiens
ggctacagcgtgcacaaggtcataacaatggcatttgctggggcaatcagttatttgttaccgtggttgataccactcgtagcactaatatgacattatgcactgaagtaactaaggaaggtacatataaaaatgataattttaaggaatatgtacgtcatgttgaagaatatgacttacagtttgtttttcagctttgcaaaattacactaactgcagagataatgacatata

SEQ ID NO: 27 - DNA, 237 bp, Homo Sapiens
ggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacataca

SEQ ID NO: 28 - DNA, 237 bp, Homo Sapiens
ggatgcaacgtgctcagggacacaataatggtatttgttggggcaatcagttatttgttactgtggtagataccacacgtagtaccaatatgtctgtttgtgctgcaattgcaaacagtgatactacatttaaaagtagtaattttaaagagtatttaagacatggtgaggaatttgatttacaatttatatttcagttatgcaaaataacattatctgcagacataatgacatata

SEQ ID NO: 29 - DNA, 237 bp, Homo Sapiens
ggttgcaacgtgcacaaggccataataatggtatttgttggagtaaccaattgtttgttactgtagttgatacaacccgtagtacaaatatgtctgtgtgttctgctgtgtcttctagtgacagtacatataaaaatgacaattttaaggaatatttaaggcatggtgaagaatatgatttacagtttatttttcagttatgtaaaataacactaacagcagatgttatgacatata

SEQ ID NO: 30 - DNA, 240 bp, Homo Sapiens
ggctacataaggcccagggccacaacaatggtatatgttggcataatcaattatttcttactgttgtggacactacccgtagtaccaactttacattatctacctctatagagtcttccataccttctacatatgatccttctaagtttaaggaatataccaggcacgtggaggagtatgatttacaatttatatttcaactgtgtactgtcacattaacaactgatgttatgtcttata

SEQ ID NO: 31 - DNA, 240 bp, Homo Sapiens
ggctgcacaaggcacagggacacaacaatggtatttgttggcataatcaattatttcttactgttgtggataccactcgcagtactaattttactttgtctactactactgaatcagctgtaccagatgtttatgatcctaataagtttaaggaatatattaggcatgttgaggaatatgatttgcaattcatatttcagttgtgtactataacattgtcaactgatgtaatgtcctata

SEQ ID NO: 32 - DNA, 240 bp, Homo Sapiens
ggctgcacaaggctcagggtttaaacaatggtatatgttggcacaatcaattgtttttaacagttgtagatactactcgcagcaccaatctttctgtgtgtgcttctactacttcttctattcctaatgtatacacacctaccagttttaaagaatatgccagacatgtggaggaatttgatttgcagtttatatttcaactgtgtaaaataacattaactacagaggtaatgtcataca

SEQ ID NO: 33 - DNA, 240 bp, Homo Sapiens
ggttacataaggcacagggtcataacaatggtgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctata

SEQ ID NO: 34 - DNA, 240 bp, Homo Sapiens
ggttacataaggcccagggccataacaatggtatttgttggcataatcagttgtttgttactgtagtggacactacccgcagtactaatttaacattatgtgcctctacacaaaatcctgtgccaagtacatatgaccctactaagtttaagcagtatagtagacatgtggaggaatatgatttacagtttatttttcagttgtgcactattactttaactgcagaggttatgtcatata

SEQ ID NO: 35 - DNA, 237 bp, Homo Sapiens
ggctccaccgtgcgcagggtcacaataatggcatttgctggaacaatcagctttttattacctgtgttgatactaccagaagtacaaatttaactattagcactgccactgctgcggtttccccaacatttactccaagtaactttaagcaatatattaggcatggggaagagtatgaattgcaatttatttttcaattatgtaaaattactttaactacagaggtaatggcttatt

SEQ ID NO: 36 - DNA, 234 bp, Homo Sapiens
ggttgcaacgtgcccaaggccataataatggcatttgctggggtaatcaattatttgttactgtagtagatactactagaagtactaacatgactattagtactgctacagaacagttaagtaaatatgatgcacgaaaaattaatcagtaccttagacatgtggaggaatatgaattacaatttgtttttcaattatgcaaaattactttgtctgcagaggttatggcatatt

SEQ ID NO: 37 - DNA, 234 bp, Homo Sapiens
ggttgcaacgtgcacagggccataataatggcatatgctggggtaatcaggtatttgttactgttgtggatactaccagaagcaccaacatgactattaatgcagctaaaagcacattaactaaatatgatgcccgtgaaatcaatcaataccttcgccatgtggaggaatatgaactacagtttgtgtttcaactttgtaaaataaccttaactgcagaagttatggcatatt

SEQ ID NO: 38 - DNA, 234 bp, Homo Sapiens
ggctgcaacgtgcccagggacataataatggcatctgttggaacaatcagttatttgtaactgttgtggataccaccaggaatacaaacatgactctttccgcaaccacacagtctatgtctacatataattcaaagcaaattaaacagtatgttagacatgcagaggaatatgaattacaatttgtgtttcaactatgtaaaatatccctgtctgctgaggttatggcctatt

SEQ ID NO: 39 - DNA, 237 bp, Homo Sapiens
ggctgcaacgtgcccagggccacaacaatggtatttgttggtttaatgaattgtttgtaaccgttgtggataccacccgcagtactaatttaaccatttgtactgctacatccccccctgtatctgaatataaagccacaagctttagggaatatttgcgccatacagaggagtttgatttgcaatttatttttcagttatgtaaaatacatttaacccctgaaattatggcctacc

SEQ ID NO: 40 - DNA, 240 bp, Homo Sapiens
ggttgcaaaaggcgcagggccacaataatggtatttgttggggaaatcagttatttgttactgttgtagatactacccgtagtacaaacatgacaatatgtgctgccactacacagtcccctccgtctacatatactagtgaacaatataagcaatacatgcgacatgttgaggagtttgacttacaatttatgtttcaattatgtagtattaccttaacggcggaggtaatggcctatc

SEQ ID NO: 41 - DNA, 234 bp, Homo Sapiens
ggctacaaaaagcccagggacataacaatggtatttgttggggtaatcaactgtttgttactgtggtagataccacacgcagtaccaacatgacattatgtgcatccgtaactacatcttccacatacaccaattctgattataaagagtacatgcgtcatgtggaagagtatgatttacaatttatttttcaattatgtagcattacattgtctgctgaagtaatggcctata

SEQ ID NO: 42 - DNA, 234 bp, Homo Sapiens
ggcttcaaaaggctcagggacataacaatggtatttgctggggaaaccacttgtttgttactgtggtagataccacacgcagtacaaatatgacactatgtgcatctgtgtctaaatctgctacatacactaattcagattataaggaatacatgcgccatgtggaggagtttgatttacagtttatttttcaattgtgtagcattacattatctgcagaagtcatggcctata